(19) United States
(12) Patent Application Publication
(10) Pub. No.: US 2004/0187383 A1
(43) Pub. Date: Sep. 30, 2004

(54) Process for carrying out a high-temperature reaction, reactor for carrying out the process, process for the scale-up of a reactor, and use

(76) Inventors: Bernd Bartenbach, Limburgerhof (DE); Michael Bachtler, Albersweiler (DE); Petra Schmitz-Bader, Frankenthal (DE); Olaf Scheidsteger, Mannheim (DE); Dieter Stapf, Brasschaat (BE)

Correspondence Address: Herbert B. Keil, KEIL & WEINKAUF, 1350 Connecticut Ave., N.W., Washington, DC, 20036, US

(21) Appl. No.: 10/806,191

(22) Filed: Mar. 23, 2004

(30) Foreign Application Priority Data
Mar. 26, 2003 (DE) 10313527.8

(51) Int. Cl. C10H 1/00; C07C 11/24; C10J 1/00

(52) U.S. Cl. 48/1; 48/2

(57) ABSTRACT

The invention relates to a process for carrying out a high-temperature reaction, in which starting materials are supplied to a reaction chamber (4) through channels (2) of a burner block (3), where in the reaction chamber (4) the high-temperature reaction having a short residence time takes place at a temperature of at least 1500° C. and the reaction mixture is subsequently rapidly cooled in a quench area (5). The cooling takes place first as a direct cooling to a temperature in the range from 650° C. to 1200° C. by supply of an evaporating quench medium and subsequently as an indirect cooling in a heat exchanger.

The invention relates to a process for carrying out a high-temperature reaction having a short residence time, in which the reaction mixture is subsequently rapidly cooled in a quench area, a reactor for carrying out the process, a process for the scale-up of a reactor, and a use.

High-temperature reactions are as a rule designated as reactions which are carried out at a temperature above 800° C. Short here is understood as meaning residence times in the millisecond range, in particular in the range from approximately 1 to 100 ms. Analogously, rapid cooling is understood as meaning a cooling in the millisecond range, in particular in the range from approximately 1 to 100 ms.

Industrially important high-temperature reactions with subsequent rapid cooling are, for example, reactions for the preparation of acetylene. A process for the preparation of acetylene by partial oxidation of hydrocarbons using oxygen is described in DE-A 44 22 815. In order to achieve the high temperatures necessary for the reaction, the starting substances natural gas and oxygen are separately preheated if possible to up to 700° C., intensively mixed in a mixing zone and reacted in a reaction chamber by means of a burner block provided with channels. The volume ratio of oxygen employed to natural gas employed here is approximately 0.6. The velocity of the oxygen/natural gas mixture in the channels of the burner block must be so high that a striking through of the flame into the mixing chamber is prevented. The reaction chamber, which is attached to the burner block, is dimensioned such that the residence time of the acetylene-containing reaction gas, of the “cleavage gas”, is only a few milliseconds. After this time, in the course of which the equilibriums corresponding to the temperature level of this reaction cannot become adjusted, the reaction products are cooled as rapidly as possible to below 300° C. using water or a residual oil in order that the acetylene formed does not decompose into soot and hydrogen. It is seen as disadvantageous in this process that the high energy of the cleavage gas cannot be further utilized.

EP-A 1 041 037 describes a “low-temperature process” for the preparation of acetylene and synthesis gas. This process has the peculiarity that temperatures of at most 1400° C. are achieved during the process, while the preparation of acetylene, as is described in DE-A 44 22 815, proceeds at a temperature of at least 1500° C. Owing to the relatively long average residence time in the reactor—as a rule at least 10 ms—the reaction mixture can be cooled by indirect cooling or by combination of direct quench and indirect cooling. It is possible by this means to utilize the heat of reaction by use of a suitable heat exchanger, for example for the generation of high-pressure steam. However, the yield and the soot formation in the low-temperature acetylene process do not always fulfill the economic requirements.

From U.S. Pat. No. 2,679,543, a process for the preparation of acetylene at a reaction temperature of 1100 to 1500° C. and residence times in the range from 0.001 to 0.05 seconds is known, according to which the reaction mixture is immediately cooled to a temperature of at most 600 to 650° C. after the reaction by direct quench and subsequently cooled to approximately 90° C. by indirect cooling. Owing to the details in the publication mentioned, it is important for the energy recovery in the subsequent indirect cooling, in the first cooling step, the direct quench, to aim for a maximum temperature which is as high as possible, but for this a maximum temperature of approximately 600 to 650° C. must not be exceeded.

It was, on the other hand, the object of the invention to make available a process for carrying out a high-temperature reaction, in which the heat of reaction is utilized to the greatest possible extent and at the same time a yield is achieved which meets economic requirements.

The object is achieved by a process for carrying out a high-temperature reaction, in which the starting materials are supplied to a reaction chamber through channels of a burner block, where in the reaction chamber the high-temperature reaction having a short residence time takes place at a temperature of at least 1500° C. and the reaction mixture is subsequently rapidly cooled in a quench area, which is characterized in that in the quench area firstly a direct cooling to a temperature in the range from 650° C. to 1200° C. takes place by supply of an evaporating quench medium and subsequently an indirect cooling in a heat exchanger takes place. The starting materials are preferably premixed.

The high-temperature reaction is in particular a reaction for the preparation of acetylene by partial oxidation of hydrocarbons using oxygen, which is advantageously carried out at a temperature in the range from 1550 to 1750° C.

It has surprisingly been found that it is possible to carry out a high-temperature reaction at high temperatures, of at least 1500° C., preferably of approximately 1550 to 1750° C., and at the same time to cool the hot reaction gas mixture in a first, direct partial quench to a temperature, which is likewise still high, in the range from 650 to 1200° C. and subsequently indirectly in a second partial quench.

It has thus been found that, compared with the process known from U.S. Pat. No. 2,679,548, a significantly lower cooling is necessary in the first partial quench and thus significantly more heat of reaction is available for the second, indirect partial quench, which can be utilized, for example, for high-pressure steam generation. It was known that the decomposition of acetylene is kinetically controlled and thus the cooling rate is decisive for the acetylene losses due to decomposition. In spite of this, it has surprisingly been found that the reaction can be carried out at a high temperature level and for the cooling in the first, direct quench relatively high maximum temperatures can be permitted without negative effects on the yield.

On account of the still high temperatures after the first quench, the water or hydrocarbon mixture which is employed as a quench medium evaporates completely. For this reason, the first quench is also described as a dry quench.

The indirect cooling in a heat exchanger in the second quench section can be utilized to generate high-pressure steam, which can be made available for further use or alternatively in order to preheat the starting materials for the reaction.

The rapid cooling takes place in the millisecond range defined at the outset, in particular in the range from approximately 1 to 100 ms, particularly preferably 1 to 50 ms. The above cooling times apply for the sum of direct and indirect cooling, the direct cooling preferably being shorter compared with the indirect cooling.

Preferably, the direct cooling takes place at a temperature in the range from 700° C. to 1000° C.

Advantageously, the direct cooling is carried out in one or more stages.

The quench medium employed for the direct cooling is advantageously water or a hydrocarbon or a hydrocarbon mixture.

The indirect cooling advantageously takes place at less than 300° C. Preferably, the indirect cooling is utilized for preheating the starting materials.

Alternatively or additionally, it is possible to utilize the indirect cooling for steam generation.

In the preparation of acetylene at high temperatures, some of the acetylene produced decomposes to give soot and hydrogen. The soot preferentially deposits on cold surfaces due to thermophoretic processes and condensation processes, in particular during the formation phase, on account of its high surface activity. This effect is particularly strong in the area of return flow zones, as occur, for example, in the toroidal areas of the burner bores.

In order to prevent such soot deposition and thus coke formation, the walls of the reaction chamber can be lined with a fire-resistant ceramic. In order that the fire-resistant ceramic is adequate for the temperatures of the high-temperature reaction, it has an alumina content of at least 80% by weight, preferably of at least 95% by weight, in particular of at least 96% by weight.

The ceramic can be introduced into the reaction chamber either in the form of stones or blocks which are already hardened and calcined or else as a cast or tamped mass which is compressed, dried and calcined only in the reaction chamber. The calcining process here preferably takes place owing to the high-temperature reaction itself.

The ceramic introduced in this way has a thickness in the range from 7 to 30 cm, preferably it has a thickness of 8 to 10 cm. Additionally, a back insulation of a ceramic having particularly good heat-insulating properties can be carried out.

Since in the direct quench the cooling medium fans out strongly after the nozzles and evaporates rapidly on account of the high temperature, the quench area cannot be arbitrarily enlarged. On account of the rapid evaporation of the cooling medium, hot streams and inhomogeneities otherwise occur in the quench area. These inhomogeneities and hot streams lead to the acetylene decomposing in the hot areas.

For this reason, the transition of the reaction chamber to the quench area is designed in the form of a gap which has a width in the range from 2 to 200 mm. In the scale-up, for a throughput enlargement the reaction chamber can thus be enlarged, where, however, the size of the gap at the transition of the reaction chamber to the quench area is to be maintained. The invention thus also relates to a process for the scale-up of a reactor, according to which for a throughput enlargement the internal diameter of the reactor is enlarged and the gap size is kept constant at the transition from the reaction chamber to the quench area.

Preferably, the transition from the reaction chamber to the quench area is restricted to a gap having a width in the range from 50 to 150 mm.

In order also to be able to integrate the gap geometry into already existing plants, the gap is preferably designed as an annular gap. A uniform flow of the reaction mixture at the transition from the reaction chamber to the quench area with an annularly designed transition is best achieved by designing the reaction chamber likewise in the form of an annular gap.

It is likewise favorable for the flow conduct if the channels in the burner block are aligned in the direction of the longitudinal axis of the reaction chamber.

In addition to the channels aligned in the longitudinal axis of the reaction chamber, some of the channels for the reaction mixture and/or channels for the supply of additional oxygen or of reaction auxiliaries can be aligned at any designed angle to the longitudinal axis of the reaction chamber.

Preferably, the quench area is constructed aligning in the direction of the longitudinal axis of the reaction chamber, in particular as a gap, particularly preferably as an annular gap. A uniform flow from the circular geometry to the annular gap geometry is achieved by the installation of a hub closure. For this, the hub closure preferably has the form of a cone or of a hemiellipsoid.

The supply of the quench medium to the direct cooling can take place in one or more stages, for which quench nozzles are attached to one or more distributors, in the case of an annular gap geometry preferably to one or more annular distributors. The quench medium can in this case be supplied to the quench area from both sides of the gap; this means in the case of an annular gap from outside and/or from inside.

The jetting in can preferably take place perpendicularly to the longitudinal axis of the quench area, where, considered in the cross-sectional plane perpendicular to the longitudinal axis of the quench area, both a radial and a tangential orientation is possible. The jetting in can also take place at an angle to the longitudinal axis of the quench area.

In the case of multistage supplies with tangential arrangement, a countercurrent positioning of the quench nozzles is preferred here.

The invention also relates to the use of the process described above or of the reactor described above for the preparation of acetylene by partial oxidation of hydrocarbons using oxygen.

The invention is explained in greater detail below with the aid of a drawing and of a working example.

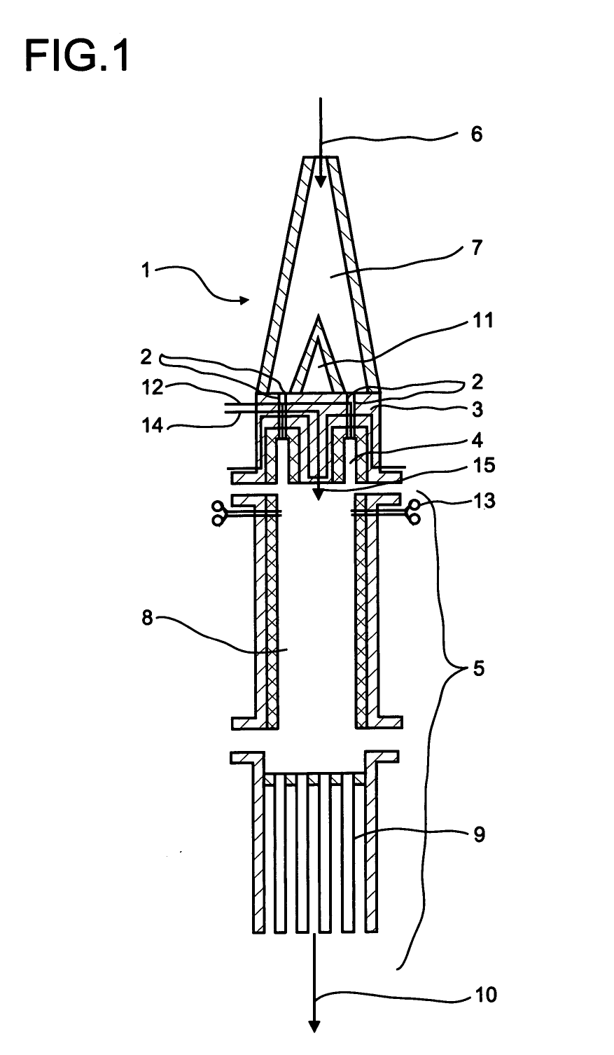

FIG. 1 shows an embodiment of a reactor according to the invention for acetylene preparation having a reaction chamber designed like an annular gap and having a first and second partial quench,

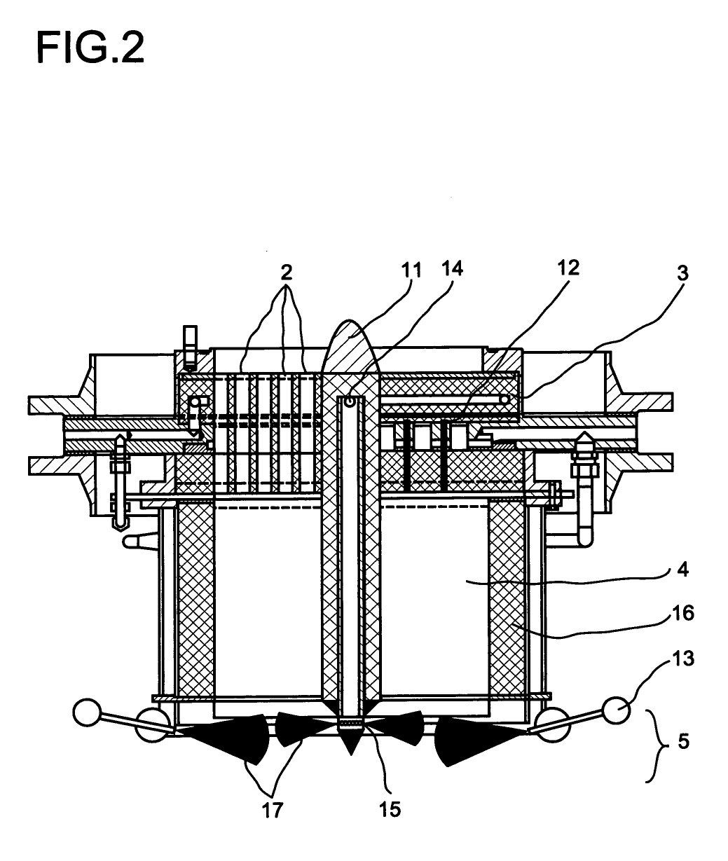

FIG. 2 shows a reaction chamber designed like an annular gap having a burner block and direct quench,

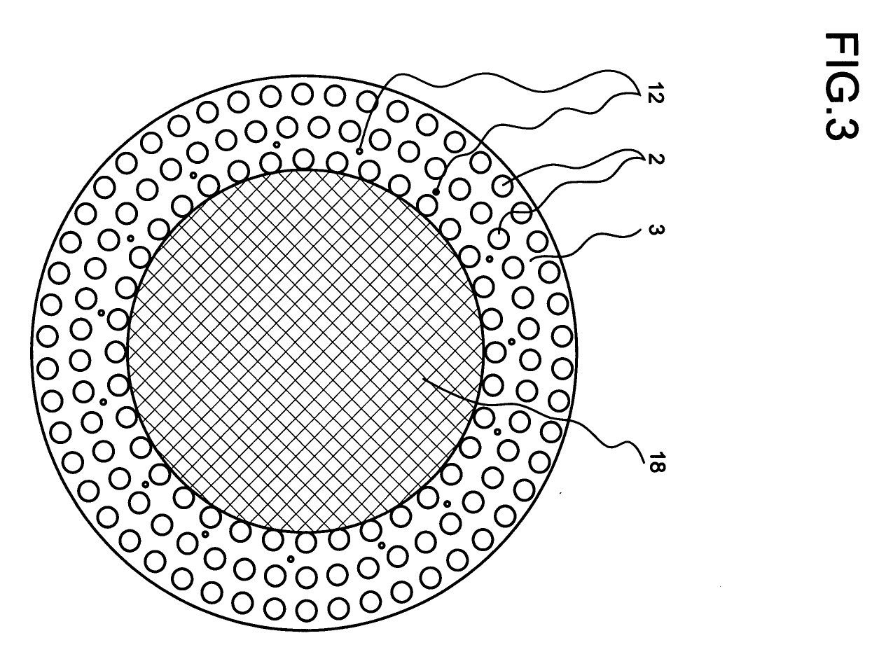

FIG. 3 shows a section through a burner block which is designed like an annular gap.

In the following, identical reference symbols designate identical or corresponding features.

FIG. 1 discloses an inventively designed reactor for acetylene preparation, having a first and second partial quench.

As starting materials, oxygen or an oxygen-containing gas and a hydrocarbon or hydrocarbon mixture, which in each case are preheated and premixed, are supplied to a reactor 1 for acetylene preparation via a delivery position 6. For the avoidance of flow separations and return flows, the mixture of oxygen or oxygen-containing gas and hydrocarbon or hydrocarbon mixture is supplied to a reaction chamber 4 via a diffuser 7 and a burner block 3 provided with channels 2. In the case of a reaction chamber 4 designed like an annular gap, in the diffuser 7 is situated a hub closure 11, which is designed such that return flows and flow separations are avoided. The preferred geometry for the hub closure 11 is a conical shape or the shape of a hemiellipsoid. In the reaction chamber 4, the reaction mixture is reacted. In order to avoid a strike back of the flame resulting here into the burner block 3 or diffuser 7 and in order to guarantee a short residence time of the reaction mixture in the reaction chamber 4, the reaction mixture flows at a high velocity. After the reaction in the reaction chamber 4, the reaction mixture arrives for cooling in a quench area 5. Here, in a first partial quench 8, a direct cooling to a temperature in the range between 650 and 1200° C., preferably to a temperature in the range from 700° C. to 1000° C., initially takes place. For the direct cooling in the first partial quench 8, the quench medium is jetted in in the first partial quench 8 via annular distributors 13 through external quench nozzles or via a line 14 and internal quench nozzles 15. After the first partial quench 8, the reaction mixture is cooled further in a second partial quench 9 to a temperature in the range from 100° C. to 300° C. The cooling can take place here in the second partial quench by means of an indirect heat exchange. The heat exchanger employed for this can be utilized, for example, for the generation of high-pressure steam or for the preheating of the starting materials. All in all, care is to be taken that the cooling phase does not exceed a time of 100 ms. In order to achieve this, the velocity of the reaction products must be chosen to be sufficiently high.

FIG. 2 shows a section from a reactor 1 for acetylene preparation, which includes the burner block 3 with channels 2 for the supply of the reaction mixture and additional channels 12 for the supply of reaction auxiliaries or additional oxygen, the reaction chamber 4 and the direct quench area in the first partial quench 8.

To avoid baking on of soot or coke, the walls of the reaction chamber 4 are lined with a fire-resistant ceramic 16. The hub closure 11, by which the reaction mixture supplied is prevented from flowing back or eddies are prevented from forming, is designed here in the form of a hemiellipsoid. The quench medium for the direct cooling is supplied to the first partial quench 8 for spraying in from outside via the quench distributor 13 and for spraying in from inside via the line 14. The quench medium leaves the external quench nozzles 15.1 and the internal quench nozzles 15.2 in the form of a spray jet 17. The amount of the quench medium is adjusted such that the quench medium completely evaporates in the spray jet 17, in order that liquid quench medium is no longer carried over and the temperature after the first partial quench 8 remains in the range from 600° C. to 1200° C.

FIG. 3 shows a cross section through a burner block 3, as is employed for the reaction chamber 4 which is like an annular gap. The burner block 3 has, concentrically arranged, the channels 2 for the supply of the reaction mixture to the reaction chamber 4. Furthermore, concentrically channels 2 for the supply of additional oxygen or reaction auxiliaries are attached. The internal area 18 of the burner block 3 designed annularly is formed from a fire-resistant ceramic.

### WORKING EXAMPLE

The function of the process according to the invention for the preparation of acetylene and synthesis gas using a first and second partial quench was investigated on a modified plant. For this, oxygen and natural gas were in each case preheated to 600° C. and continuously mixed and reacted in a volumetric ratio of 0.59. The flame forming was cooled shortly after the main reaction zone to a temperature of 850° C. in about 10 to 50 ms by jetting in water via a concentric ring of nozzles. The water jetted in evaporated completely here. After the jetting in of the water, the product gas mixture was cooled to about 200° C. by means of an indirect heat exchanger with the generation of high-pressure steam. After the condensing out of the water, the product gas mixture contained 7.9% of acetylene, 3.4% of carbon dioxide, 5.5% of methane, 25.2% of carbon monoxide and 56.4% of hydrogen. The yield was thus about 29% based on carbon. For comparison, the Sachsse-Bartholome process employed in production can be used, in which a yield of 29.5% of acetylene, based on carbon, is achieved under identical boundary conditions.

### List of Reference Symbols

1. reactor

2. channels

3. burner block

4. reaction chamber

5. quench area

6. delivery position

7. diffuser

8. first partial quench

9. second partial quench

10. outlet

11. hub closure

12. addition channels

13. quench distributor

14. line

15.1 external quench nozzles

15.2 internal quench nozzles

16. fire-resistant ceramic

17. spray jet

18. internal area

We claim:

1. A process for carrying out a high-temperature reaction, in which starting materials are supplied to a reaction chamber (4) through channels (2) of a burner block (3), where in the reaction chamber (4) the high-temperature reaction having a short residence time takes place at a temperature of at least 1500° C. and the reaction mixture is subsequently rapidly cooled in a quench area (5), characterized in that in the quench area (5) firstly a direct cooling to a temperature in the range from 650° C. to 1200° C. takes place by supply of an evaporating quench medium and subsequently an indirect cooling in a heat exchanger takes place.

2. A process as claimed in claim 1, characterized in that the starting materials are premixed.

3. A process as claimed in claim 1, characterized in that the direct cooling takes place to a temperature in the range from 700° C. to 1000° C.

4. A process as claimed in claim 1, characterized in that the direct cooling takes place in one or more stages.

5. A process as claimed in claim 1, characterized in that the quench medium is water or a hydrocarbon or a hydrocarbon mixture.

6. A process as claimed in claims 1, characterized in that the indirect cooling takes place to less than 300° C.

7. A process as claimed in claim 1, characterized in that the indirect cooling is utilized for the preheating of the starting materials or for the generation of steam.

8. A reactor (1) for carrying out a process as claimed in claim 1, characterized in that all the surfaces restricting the reaction chamber (4) are formed using a fire-resistant ceramic stable at reaction temperature having an alumina content of at least 80%.

9. A reactor (1) as claimed in claim 8, characterized in that the fire-resistant ceramic is introduced into the reaction chamber (4) in the form of stones or blocks or as a cast or tamped mass and subsequently compressed, dried and calcined, the calcining process preferably taking place owing to the high temperature reaction.

10. A reactor (1) as claimed in claim 8, characterized in that the fire-resistant ceramic has a thickness in the range from 7 to 30 cm.

11. A reactor (1) as claimed in claim 8, characterized in that the transition of the reaction chamber (4) to the quench area (5) is designed in the form of a gap which has a width in the range from 2 to 200 mm.

12. A reactor (1) as claimed in claim 11, characterized in that the transition of the reaction chamber (4) to the quench area (5) is designed in the form of an annular gap.

13. A reactor (1) as claimed in claim 11, characterized in that the reaction chamber (4) is designed in the form of an annular gap.

14. A reactor (1) as claimed in claim 11, characterized in that the channels (2) in the burner block (3) are aligned in the direction of the longitudinal axis of the reaction chamber (4).

15. A reactor (1) as claimed in claim 11, characterized in that some of the channels (2) for the reaction chamber and/or channels (6) for the supply of additional oxygen or of reaction auxiliaries are aligned at any desired angle to the longitudinal axis of the reaction chamber (4).

16. A reactor (1) as claimed in claim 11, characterized in that the quench area (5) is constructed aligning in the direction of the longitudinal axis of the reaction chamber (4).

17. A reactor (1) as claimed in claim 8, characterized in that the supply of the quench medium to the direct cooling takes place via quench nozzles which are attached to one or more distributors.

18. A reactor (1) as claimed in claim 17, characterized in that the quench nozzles are arranged radially or tangentially to the main flow direction of the reaction mixture, where in the case of multistage supplies with tangential arrangement a countercurrent positioning of the quench nozzles is preferred.

19. A process for the scale-up of a reactor (1) as claimed in claim 11, characterized in that for a throughput enlargement the internal diameter of the reactor (1) is enlarged and the gap size at the transition from the reaction chamber (4) to the quench area (5) is kept constant.

20. The use of a process as claimed in claim 1 or of a reactor (1) as claimed in claim 8 for the preparation of acetylene by partial oxidation of hydrocarbons using oxygen.
